(19) United States
(12) Patent Application Publication
(10) Pub. No.: US 2024/0190801 A1
(43) Pub. Date: Jun. 13, 2024

(54) PROCESS FOR THE PURIFICATION OF VANILLIN OR A VANILLIN DERIVATIVE OBTAINED BY A BIOTECHNOLOGICAL PROCESS

(71) Applicant: SPECIALTY OPERATIONS FRANCE, Lyon (FR)

(72) Inventors: Talsida Zougmore, Francheville (FR); Rachid Djillali, Lyon (FR); Laurent Garel, Lyon (FR)

(73) Assignee: SPECIALTY OPERATIONS FRANCE, Lyon (FR)

(21) Appl. No.: 18/550,801

(22) PCT Filed: Mar. 14, 2022
(86) PCT No.: PCT/EP2022/056527

§ 371 Date: Sep. 15, 2023

(30) Foreign Application Priority Data
Mar. 15, 2021 (FR) 2102534

(51) Int. Cl. C07C 45/81; C07C 45/79; C07C 45/80; C12P 7/24

(52) U.S. Cl. CPC C07C 45/81; C07C 45/79; C07C 45/80; C12P 7/24

(57) ABSTRACT

The present invention relates to a process for purifying vanillin or derivatives thereof obtained via a biotechnological process, comprising at least one step in which vanillin or derivatives thereof are separated from vanillyl alcohol or derivatives thereof by crystallization.

### FIELD OF THE INVENTION

The present invention relates to a process for purifying vanillin or a vanillin derivative obtained via a biotechnological process.

### PRIOR ART

Vanillin may be obtained by various methods known to those skilled in the art, notably by the following two routes:
a “natural” route based on a biotechnological process notably comprising the cultivation of a microorganism that is capable of enabling the biotransformation of a fermentation substrate into vanillin. Such a process, in which the fermentation substrate is ferulic acid, is notably known from patent application EP 0885968. U.S. Pat. No. 5,017,388 describes a process in which the fermentation substrate is eugenol and/or isoeugenol. These processes lead to the preparation of a vanillin known as natural vanillin.
a “synthetic” route comprising conventional chemical reactions starting from guaiacol and not involving microorganisms. This process leads to the preparation of a vanillin known as synthetic vanillin.

Finally, vanillin can also be prepared via a “biobased” route, in which vanillin is derived from lignin. In particular, mention may be made of U.S. Pat. No. 2,745,796, DE 1132113 and the article entitled “Preparation of lignin from wood dust as vanillin source and comparison of different extraction methods” by Azadbakht et al. in International Journal of Biology and Biotechnology, 2004, vol. 1, No. 4, pages 535-537.

At the present time, natural vanillin can be purified via the process described in patent application EP 2791098, which includes a step of liquid/liquid extraction of impurities with a pKa higher than that of vanillin. The yield for this process is good, generally greater than 80%, but in order to obtain improved organoleptic properties such as the odor and/or color of vanillin, several additional purification steps are required, thus causing the overall yield of this process to fall. The overall energy efficiency of this process is also degraded due to the use of large amounts of solvents.

International patent application WO 2014/114590 also describes a process for purifying natural vanillin. This process consists in evaporating natural vanillin, which may be performed by distillation or by vacuum evaporation of molten vanillin. This process is capable of producing very pure natural vanillin, in good yield, with a device that is simple to use and which operates continuously so as to be compatible with industrial processes. However, such a process may be difficult to implement due to the number and size of the items of equipment required.

Moreover, vanillin or derivatives thereof obtained via a biotechnological process may contain certain impurities with boiling points very close to that of vanillin or derivatives thereof. Thus, it is necessary to size the equipment suitably so as to enable efficient separation of vanillin or derivatives thereof from these products. This usually involves extending the residence time in the distillation equipment, which may generate new impurities due to the high-temperature instability of vanillin and/or impurities.

For this reason, it would be advantageous to have an improved process relative to those proposed in the prior art, notably in terms of environmental and/or energy impact, while at the same time improving the overall purification yield and also the yield of vanillin or derivatives thereof. It is also important that the vanillin purification process allows the production of a vanillin whose organoleptic properties are preserved notably in terms of taste, color and/or odor.

### BRIEF DESCRIPTION

The present invention relates to a process for purifying vanillin or derivatives thereof obtained via a biotechnological process, comprising at least one step in which vanillin or derivatives thereof are separated from vanillyl alcohol or derivatives thereof.

### DESCRIPTION OF THE FIGURES

FIGS. 1 to 5 schematically show various processes for purifying vanillin or derivatives thereof obtained via a biotechnological process according to the present invention.

### DETAILED DESCRIPTION

In the context of the present invention, and unless otherwise indicated, the expression “between . . . and . . . ” includes the limits.

In the present invention, the term “natural vanillin” denotes a vanillin obtained via a biotechnological process. Thus, a process for preparing natural vanillin denotes herein a biotechnological process comprising the cultivation of a microorganism that is capable of enabling the transformation of a fermentation substrate into vanillin. The microorganism may be of wild-type origin or may be a genetically modified microorganism (GMM) obtained by molecular biology. Very preferentially, it may be a ferulic acid fermentation process, such as that described in patent application EP 0885968. According to a particular aspect, vanillin may be produced via a glucose or protocatechuic acid fermentation process as described in patent application WO 2013/022881.

In the context of the present invention, the term “vanillin derivative” refers to any compound that may be derived from vanillin and in particular to vanillin in salified form or glucovanillin.

In the context of the present invention, the term “vanillyl alcohol derivative” refers to any compound that may be derived from vanillyl alcohol and in particular to vanillyl alcohol in salified form or to vanillyl alcohol glucoside.

In the context of the present invention, the term “crystallization” refers to a process in which a substance passes into the solid state via a physical process, notably such as lowering the temperature.

In the context of the present invention, the term “precipitation” refers to a process in which a substance passes into the solid state via a chemical transformation, notably such as protonation through a change in pH.

The present invention relates to a process for purifying vanillin or derivatives thereof obtained via a biotechnological process, comprising at least one step in which vanillin or derivatives thereof are separated from vanillyl alcohol or derivatives thereof.

According to the present invention, the purification process of the present invention comprises at least one step of crystallizing vanillin or derivatives thereof. The crystallization step allows the purification of vanillin or derivatives thereof, obtained via a biotechnological process, to a purity of between 85% and 99%, preferably greater than or equal to 90%, very preferentially greater than or equal to 95%. Advantageously, the crystallization process allows the production of purified vanillin or derivatives thereof with a purity of greater than or equal to 95%, preferably greater than or equal to 97%, very preferentially greater than or equal to 99%. Vanillin with a purity of between 85% and 99% generally comprises at least one other compound chosen from vanillyl alcohol, vanillic acid, guaiacol, acetovanillone, 4-((4-hydroxy-3-methoxybenzyl)oxy)-3-methoxybenzaldehyde and 4-hydroxy-3-(4-hydroxy-3-methoxybenzyl)-5-methoxybenzaldehyde. The vanillyl alcohol content is generally between 0.01% and 15% by weight.

The crystallization is generally performed in an alcoholic solution. Preferably, the solvent used for the crystallization may be a water-soluble alcohol, preferably ethanol. The solvent used for the crystallization may be a water/alcohol mixture. In general, the amount of alcohol is between 2% and 40% by mass, preferably between 5% and 35% by mass, and very preferentially between 15% and 25% by mass. In general, during crystallization, the vanillin concentration at the start of crystallization is between 5% and 60% by weight, preferably between 10% and 50% by weight, advantageously between 15% and 35% by weight, and even more preferentially between 15% and 25%. Very advantageously, crystallization allows the separation of vanillin or derivatives thereof from vanillyl alcohol or derivatives thereof, and this separation is advantageously performed without degradation of the vanillin or derivatives thereof. Preferably, crystallization allows the separation of vanillin and vanillyl alcohol, and this separation is advantageously performed without degrading the vanillin. The crystallization is performed at a temperature of between 0° C. and 50° C. The crystallization yield is generally greater than or equal to 80%. The vanillin obtained on conclusion of the crystallization step generally has a color in ethanolic solution at 10% by weight of less than or equal to 150 Hazen, preferably less than or equal to 100 Hazen, and very preferentially less than or equal to 50 Hazen.

The present invention relates to a process for purifying vanillin or derivatives thereof obtained via a biotechnological process in which vanillin or derivatives thereof are separated from vanillyl alcohol or derivatives thereof. Advantageously, the process for purifying vanillin or derivatives thereof is particularly optimized and allows the amount of organic solvent used to be reduced. In general, the amount of organic solvent used in the purification process is reduced by at least 5% relative to the processes for purifying vanillin obtained via a biotechnological process. The solvent reduction is calculated relative to processes using at least one organic solvent. The amount of solvent used is calculated from any amount of solvent used for purification between the fermentation must and up to the production of purified, crystallized vanillin. The process is thus improved relative to the prior art processes in that it has a reduced ecological and/or environmental impact.

The present invention relates to a process for purifying vanillin or derivatives thereof obtained via a biotechnological process in which vanillin or derivatives thereof are separated from vanillyl alcohol or derivatives thereof. Advantageously, the process of the present invention allows the upgrading of vanillyl alcohol or derivatives thereof. Indeed, vanillyl alcohol or derivatives thereof may be recovered in the filtrate on conclusion of the crystallization. Unlike prior art processes, this vanillyl alcohol or derivatives thereof may then be upgraded as synthetic intermediates, notably for the preparation of organic compounds for the cosmetics and perfumery industries. The process of the present invention does not degrade the vanillyl alcohol or derivatives thereof.

In the context of the present invention, on conclusion of a biotechnological process for the preparation of vanillin or derivatives thereof, the vanillin or derivatives thereof are in aqueous solution in a fermentation must. Thus, the process of the present invention consists in purifying an aqueous solution of vanillin or derivatives thereof. In general, the concentration of vanillin in the aqueous solution is between 0.2% and 5% by weight, preferably between 0.8% and 2.5% by weight, very preferentially between 1.0% and 1.8% by weight. The aqueous solution also comprises:
biomass: in general, the amount of biomass is between 0.5% and 5% by weight of dry matter, preferably between 1.0% and 2.0% by weight of dry matter;
vanillyl alcohol: in general, the amount of vanillyl alcohol is between 0.01% and 0.5% by weight, preferably between 0.05% and 0.3% by weight;
vanillic acid: in general, the amount of vanillic acid is between 0.01% and 0.5% by weight, preferably between 0.05% and 0.3% by weight.

The aqueous solution may also comprise other compounds such as ferulic acid and ferulic acid derivatives, coumaric acid, para-hydroxybenzaldehyde and guaiacol. These compounds may be separated from the vanillin or derivatives thereof via the process of the present invention. Advantageously, at least one compound chosen from ferulic acid, ferulic acid derivatives, coumaric acid, para-hydroxybenzaldehyde or guaiacol may be upgraded notably as a synthetic intermediate.

The purification process of the present invention may comprise at least one step consisting in separating the biomass from the aqueous solution of vanillin or derivatives thereof. This step is a step of separating a solid phase: the biomass, from a liquid phase. The liquid phase obtained on conclusion of the biomass separation step is an aqueous phase comprising vanillin or derivatives thereof.

According to one aspect, the biomass separation step may be performed by filtration such as frontal filtration or tangential filtration, in particular membrane filtration, such as microfiltration, ultrafiltration, nanofiltration, or reverse osmosis. Membrane filtration may be performed by concentration or diafiltration. Advantageously, when the biomass separation step is frontal filtration, adjuvants may be added so as to improve the filtration efficacy.

In order to improve the solid/liquid separation efficacy, it is possible to perform several solid/liquid separation steps, notably to remove the smallest solid particles.

According to a particular aspect, the biomass separation step may be performed with one or more microfiltration membranes, in particular having a cut-off limit of about 0.2 μm, followed by one or more ultrafiltrations having a cut-off limit smaller than that of the microfiltration. In this configuration, the vanillin is dissolved in the aqueous phase and the biomass is retained by the membranes. Advantageously, ultrafiltration also allows the separation of molecules dissolved in the fermentation must.

In order to improve the yield of vanillin or derivatives thereof in the aqueous phase, a solvent, preferably water, is added during the filtration step. In general, the amount of solvent added is between 0.5 and 5 volume equivalents of fermentation must.

According to one aspect, microfiltration, ultrafiltration or diafiltration may be coupled with a nanofiltration or reverse osmosis step. Nanofiltration allows the concentration of vanillin in the retentate of the nanofiltration step to be increased, while the water passes through the membrane (permeate of the nanofiltration step). The permeate from this nanofiltration step may advantageously be recycled. In general, reverse osmosis has a cut-off limit of less than or equal to 100 Da. In general, nanofiltration has a cut-off limit of less than or equal to 400 Da. In general, nanofiltration has a cut-off limit of greater than or equal to 100 Da, for example a cut-off limit of between 100 and 250 Da.

Prior to this step of separating the biomass from the aqueous solution of vanillin or derivatives thereof, the fermentation must may be stabilized.

In the context of the present invention, the term “stabilization” refers to any method for preventing the degradation, notably by reduction, of vanillin or derivatives thereof between the end of fermentation and the purification process.

According to a first aspect, stabilization may be performed by the addition of at least one compound. The compound is preferentially chosen from sodium benzoate, ascorbic acid and salts thereof, potassium, calcium or sodium sorbate, zinc sulfate, propanoic acid, acetic acid or salts thereof, or sodium diacetate. Preferably, the amount of compound added is between 0.2 g/L and 6 g/L.

According to another aspect, stabilization may be performed by changing the temperature of the fermentation must. In general, the temperature is controlled so as to achieve a temperature of between 15° C. and 23° C., preferably between 18° C. and 21° C.

According to another aspect, stabilization may be performed by changing the pH of the fermentation must. In general, the pH is controlled so as to achieve a pH of less than or equal to 7.5, preferably less than or equal to 7, very preferentially less than or equal to 6.8. In general, the pH is controlled to achieve a pH of greater than or equal to 5.0, preferably greater than or equal to 6.

According to another aspect, the fermentation must may also be pasteurized. In general, the fermentation must is then heated to a temperature of between 50° C. and 90° C., preferably between 60° C. and 80° C. Heating is generally maintained for between 10 min and 120 min, preferably between 15 min and 45 min, for example for 20 min.

According to another aspect, the fermentation must may be stabilized by ultrasound. In general, ultrasound is emitted into the fermentation must for a period of between 10 min and 120 min.

According to a particular aspect, these aspects may be performed separately or in combination; thus, by way of nonlimiting example, it is possible to modify the temperature, the pH and to add a compound under the conditions described above.

Among these methods, methods without adding compounds are advantageous: without wishing to be bound by any theory, they allow possible deleterious effects on vanillin or derivatives thereof, notably in terms of odor or color, to be avoided.

However, the addition of certain compounds chosen for their compatibility with vanillin or derivatives thereof and the absence of side effects on vanillin or derivatives thereof may prove particularly advantageous in facilitating the subsequent process for purifying vanillin or derivatives thereof.

The purification process generally includes at least one liquid/liquid extraction step allowing the recovery of vanillin or derivatives thereof in an organic solution. The liquid/liquid extraction step advantageously allows the vanillin or derivatives thereof in aqueous solution in the fermentation must or, on conclusion of a solid/liquid separation of the fermentation must, to be transferred into an organic phase. Thus, on conclusion of the liquid/liquid extraction step, an organic phase comprising vanillin or derivatives thereof is obtained. The liquid/liquid extraction step advantageously allows the separation of vanillin or derivatives thereof from highly water-soluble compounds such as vanillic acid or derivatives thereof, or ferulic acid.

In general, the pH of the aqueous solution is between 5 and 8, preferably between 5.5 and 7.5, very preferentially between 6 and 7.

In general, the extraction solvent is chosen for its capacity for dissolving vanillin or derivatives thereof; advantageously, the solvent may be of biobased origin. According to one aspect, the chosen solvent is compatible with food industry standards, notably FEMA GRAS, and is water-immiscible. Preferably, the solvent is chosen from methyl ethyl ketone, methyl isobutyl ketone, ethyl acetate, isopropyl acetate or mixtures thereof.

The liquid/liquid extraction step may be a discontinuous liquid/liquid extraction. In order to maximize the amount of vanillin or derivatives thereof obtained in the organic phase, the volume ratio of solvent relative to the aqueous solution of vanillin or derivatives thereof is between 1:5 and 5:1, preferably between 1:1 and 5:1, preferably between 1.5:1 and 3:1.

According to another aspect, the liquid/liquid extraction step may be performed continuously. In general, the volume ratio of solvent relative to the aqueous solution of vanillin or derivatives thereof is between 5:1 and 1:2, preferably between 3:1 and 1:1.

The yield of vanillin or derivatives thereof from the liquid/liquid extraction step is generally greater than or equal to 95%, preferably greater than or equal to 97%, very preferentially greater than or equal to 98%.

According to a particular aspect, the biomass separation and liquid/liquid extraction steps may be performed simultaneously. This particular aspect allows vanillin or derivatives thereof to be extracted into the organic solvent from the fermentation must. The biomass is then separated from the two-phase system. This process is advantageous in that the loss of vanillin or derivatives thereof in the biomass is reduced. This separation step may notably be performed continuously by centrifugation, in particular using a two-phase or three-phase centrifuge or a countercurrent extractor.

The concentration (C) of the organic solution of vanillin or derivatives thereof obtained on conclusion of the liquid/liquid extraction step is generally between 0.1% by weight and 10% by weight.

Optionally, a stripping step may be performed on the aqueous stream obtained on conclusion of the liquid/liquid extraction step. Advantageously, the stripping step allows the recovery of organic compounds contained in the aqueous stream, notably vanillin or derivatives thereof. Thus, stripping enables the yield of vanillin or derivatives thereof to be improved. The stripping step also facilitates the treatment of the aqueous effluents.

The process of the present invention comprises at least one step of concentrating an aqueous or organic solution of vanillin or derivatives thereof.

According to one particular aspect, the concentrating step may be performed on an aqueous solution of vanillin or derivatives thereof obtained on conclusion of the biomass separation step. According to another aspect, the concentrating step may be performed on an organic solution of vanillin or derivatives thereof obtained on conclusion of the liquid/liquid extraction step. In general, on conclusion of the concentrating step, vanillin or derivatives thereof are in liquid or solid form, depending on the temperature. In general, at least 95% of the solvent has been separated from the vanillin or derivatives thereof. However, the concentrating step may consist of one or more concentrating steps.

Thus, according to a particular aspect, at least 95% of the amount of solvent present in the organic solution of vanillin or derivatives thereof obtained on conclusion of the liquid/liquid extraction may be evaporated.

According to another aspect, the concentrating of the organic solution obtained on conclusion of the liquid/liquid extraction step may be partial, to result in the formation of a concentrated solution of vanillin or derivatives thereof (C1), preferably having a higher concentration than the concentration (C) of the organic solution of vanillin or derivatives thereof obtained on conclusion of the liquid/liquid extraction step.

According to a particular aspect, the organic solution of vanillin or derivatives thereof obtained on conclusion of the liquid/liquid extraction step is concentrated so as to obtain an organic solution of vanillin or derivatives thereof (C1) having a concentration of between 10% by weight and 95% by weight.

The organic solution of vanillin or derivatives thereof (C1) may be subjected to one or more subsequent concentrating steps so as to obtain vanillin or derivatives thereof in liquid or solid form as a function of the temperature.

The concentrating step may be performed in a continuous or batch process.

In a batch process, the concentrating step may be performed in a stirred reactor or in a distillation column. Preferentially, the concentrating step is performed under vacuum, preferably at a pressure of between 5 mbar and 300 mbar, more preferably between 50 mbar and 250 mbar. The concentrating step is generally performed at a temperature of between 25° C. and 100° C.

In a continuous process, the concentrating step may be performed using tray or packed distillation columns, falling-film evaporators, scraped-film evaporators or baffle columns.

In general, the concentrating step is performed at a distillation head pressure of between 0.5 bar and 5 bar, preferably between 1 bar and 3 bar. The temperature at the distillation head is generally between 75° C. and 150° C., preferably between 85° C. and 120° C. and very preferably between 90° C. and 110° C.

Advantageously, the concentrating step is performed so as to reduce the residence time of vanillin or derivatives thereof in the reactors or columns.

The concentrating step allows the vanillin or derivatives thereof to be recovered at the bottom of the column, while the solvent is recovered at the top. The vanillin or derivatives thereof at the bottom of the column generally have a purity of greater than or equal to 90%. Other compounds are present with vanillin or derivatives thereof at the bottom of the column, notably vanillyl alcohol or derivatives thereof.

The purification process of the present invention may comprise at least one step in which vanillin or derivatives thereof are separated from compounds having a higher boiling point than that of vanillin or derivatives thereof. This step may be a de-tarring step.

The de-tarring step may be performed in a distillation column, a baffle column, a falling-film evaporator, or a scraped-film evaporator.

The purification process of the present invention includes at least one step of crystallizing vanillin or derivatives thereof as described previously. Crystallization may notably be performed on vanillin or derivatives thereof obtained on conclusion of at least one step chosen from liquid/liquid extraction, concentration and de-tarring.

The purification process of the present invention may also comprise a step of precipitating vanillin or derivatives thereof in the presence of biomass. This precipitation step is generally prior to the biomass separation step. Precipitation of vanillin or derivatives thereof in the presence of biomass is generally performed at a temperature of between 0° ° C. and 6° C. This step may be simultaneous with a step of stabilizing the fermentation must, notably by modifying the pH. This aspect enables the subsequent separation of the biomass from the liquid phase. Since vanillin or derivatives thereof are in solid form, they are recovered in the cake with the biomass. The cake is advantageously taken up with a solvent in which the vanillin or derivatives thereof are soluble, to allow the formation of a solid phase and a liquid organic phase of vanillin or derivatives thereof which may be purified according to the various concentrating, de-tarring and/or crystallization steps as described previously.

According to another aspect of the present invention, the process for purifying vanillin or derivatives thereof obtained via a biotechnological process is characterized in that no organic solvent is used with the exception of the solvent used during crystallization. Thus, according to a particular aspect of the present invention, the amount of organic solvent used is reduced by 100% relative to the processes of the prior art.

According to a particular aspect, the process for purifying vanillin or derivatives thereof obtained via a biotechnological process and, prior to the crystallization step, may comprise at least one solid/liquid separation, concentrating and/or de-tarring step as described previously without the use of organic solvent.

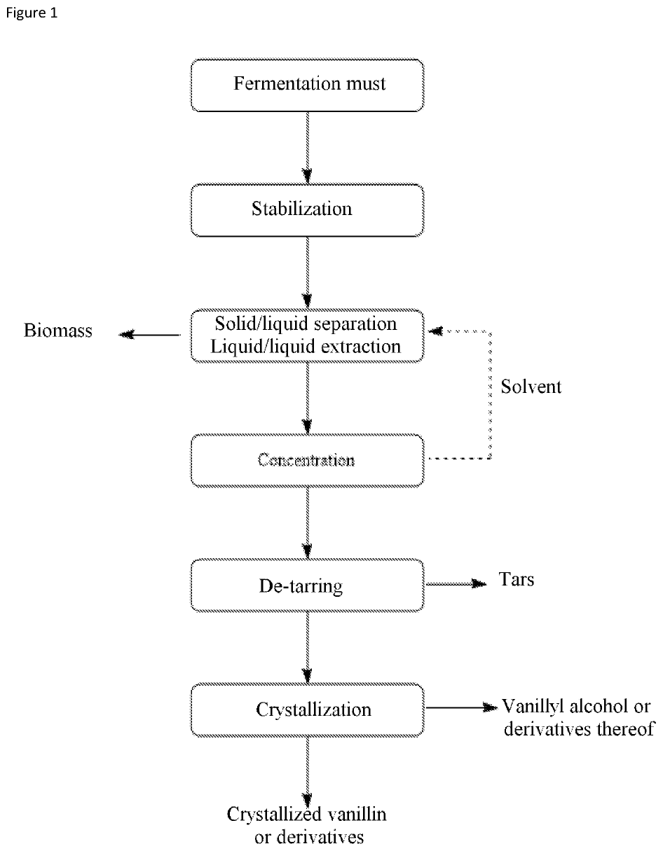

According to a first embodiment represented by FIG. 1, the fermentation must is subjected to a stabilization step. This stabilized fermentation must is then centrifuged with a plate centrifuge allowing biomass separation. At the same time, an organic solvent is added to allow extraction of the vanillin into organic solution, and the aqueous and organic phases are separated. The organic phase is then subjected to concentration, preferably using a scraped-film evaporator and/or a falling-film evaporator. The evaporated organic solvent is recycled into the biomass separation step. The vanillin is then subjected to a second evaporation step, preferably in a scraped-film evaporator so as to separate the vanillin from the heavy products. Vanillin is recovered at the top of the evaporator, along with any impurities such as vanillyl alcohol. Finally, this stream recovered at the evaporator head is crystallized to allow the preparation of a crystallized vanillin having a purity of greater than or equal to 99.5% and having a color in ethanolic solution at 10% by weight of less than or equal to 150 Hazen, preferably less than or equal to 100 Hazen, and very preferentially less than or equal to 50 Hazen.

According to a second embodiment, also represented by FIG. 1, the fermentation must is subjected to a stabilization step. This stabilized fermentation must is then decanted and filtered, allowing the biomass to be separated. At the same time, an organic solvent is added to allow extraction of the vanillin into organic solution, and the aqueous and organic phases are separated. The organic phase is then concentrated by vacuum distillation. The evaporated organic solvent may be recycled into the biomass separation step. The vanillin is then subjected to a second evaporation step, preferably in a scraped-film evaporator so as to separate the vanillin and the heavy products. The vanillin is recovered at the top of the evaporator, along with any impurities such as vanillyl alcohol. Finally, this stream recovered at the evaporator head is crystallized to allow the preparation of a crystallized vanillin having a purity of greater than or equal to 99.9% and having a color in ethanolic solution at 10% by weight of less than or equal to 150 Hazen, preferably less than or equal to 100 Hazen, and very preferentially less than or equal to 50 Hazen.

According to a third embodiment, represented by FIG. 1, the fermentation must is subjected to a stabilization step by lowering the temperature to 21° ° C., adjusting the pH to 6.8, and adding an adjuvant or pasteurizing at 60° C. for 20 min. An organic solvent is simultaneously added to allow extraction of the vanillin into organic solution. Separation of the biomass from the aqueous and organic phases is performed using a countercurrent extractor. The organic phase is then concentrated by vacuum distillation. The evaporated organic solvent may be recycled into the biomass separation step. The vanillin is then subjected to a second evaporation step, preferably in a scraped-film evaporator so as to separate the vanillin and the heavy products. The vanillin is recovered at the top of the evaporator, along with any impurities such as vanillyl alcohol. Finally, this stream recovered at the evaporator head is crystallized to allow the preparation of a crystallized vanillin having a purity of greater than or equal to 99.9% and having a color in ethanolic solution at 10% by weight of less than or equal to 150 Hazen, preferably less than or equal to 100 Hazen, and very preferentially less than or equal to 50 Hazen.

According to a fourth embodiment, represented by FIG. 1, the fermentation must is subjected to a stabilization step. This stabilized fermentation must is stirred with simultaneous addition of an organic solvent. This mixture is then separated using a centrifugal decanter. The organic phase is then concentrated by continuous or discontinuous vacuum distillation. The evaporated organic solvent may be recycled into the biomass separation step. The vanillin is then subjected to a second evaporation step, preferably in a scraped-film evaporator so as to separate the vanillin and the heavy products. The vanillin is recovered at the top of the evaporator, along with any impurities such as vanillyl alcohol. Finally, this stream recovered at the evaporator head is crystallized to allow the preparation of a crystallized vanillin having a purity of greater than or equal to 99.9% and having a color in ethanolic solution at 10% by weight of less than or equal to 150 Hazen, preferably less than or equal to 100 Hazen, and very preferentially less than or equal to 50 Hazen.

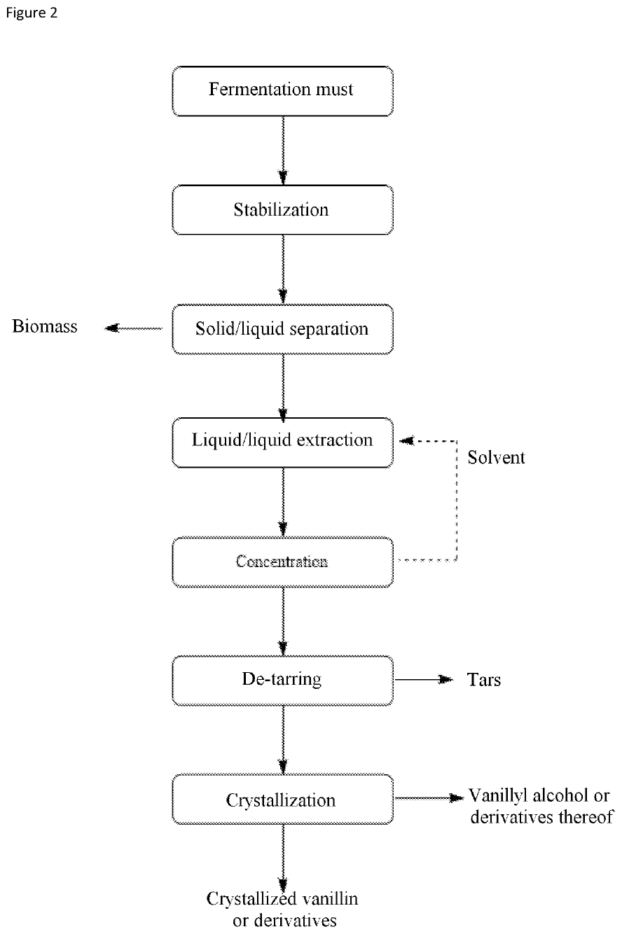

According to a fifth embodiment, represented by FIG. 2, the fermentation must is subjected to a stabilization step. This stabilized fermentation must is filtered, allowing the biomass to be separated. Filtration is performed by microfiltration followed by nanofiltration. An organic solvent is then added to allow the vanillin to be extracted into organic solution, and the aqueous and organic phases are separated by continuous extraction. The organic phase is then concentrated by continuous or discontinuous vacuum distillation. The evaporated organic solvent may be recycled into the biomass separation step. The vanillin is then subjected to a second evaporation step, preferably in a scraped-film evaporator so as to separate the vanillin and the heavy products. The vanillin is recovered at the top of the evaporator, along with any impurities such as vanillyl alcohol. Finally, this stream recovered at the evaporator head is crystallized to allow the preparation of a crystallized vanillin having a purity of greater than or equal to 99.9% and having a color in ethanolic solution at 10% by weight of less than or equal to 150 Hazen, preferably less than or equal to 100 Hazen, and very preferentially less than or equal to 50 Hazen.

According to a sixth embodiment, represented by FIG. 2, the fermentation must is subjected to a stabilization step. This stabilized fermentation must is filtered, allowing the biomass to be separated by frontal filtration. An organic solvent is then added to allow the vanillin to be extracted into organic solution, and the aqueous and organic phases are separated by continuous extraction. The organic phase is then concentrated by continuous or discontinuous vacuum distillation. The evaporated organic solvent may be recycled into the biomass separation step. The vanillin is then subjected to a second evaporation step, preferably in a scraped-film evaporator so as to separate the vanillin and the heavy products. The vanillin is recovered at the top of the evaporator, along with any impurities such as vanillyl alcohol. Finally, this stream recovered at the evaporator head is crystallized to allow the preparation of a crystallized vanillin having a purity of greater than or equal to 99.9% and having a color in ethanolic solution at 10% by weight of less than or equal to 150 Hazen, preferably less than or equal to 100 Hazen, and very preferentially less than or equal to 50 Hazen.

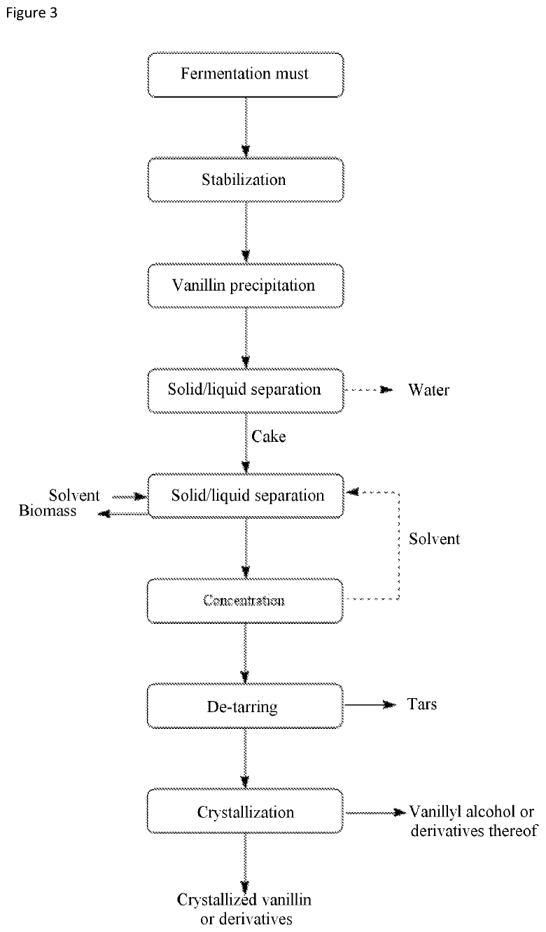

According to a seventh embodiment represented by FIG. 3, the fermentation must is stabilized and precipitated. A solid/liquid separation step is then performed to separate the cake comprising vanillin and biomass from a liquid aqueous phase comprising certain impurities from the fermentation process. The vanillin contained in the cake is then dissolved with an organic solvent or water to allow separation of an aqueous or organic liquid phase comprising the vanillin from the biomass. The aqueous or organic liquid phase comprising vanillin is then subjected to the concentrating, separation of the heavy products and crystallization steps as described in the first embodiment.

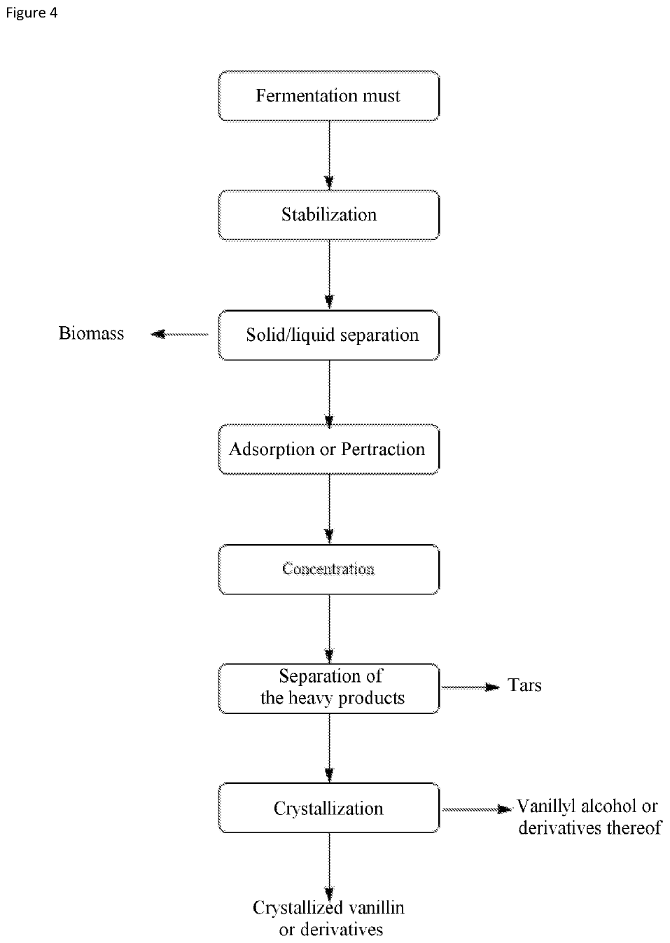

According to an eighth embodiment, represented by FIG. 4, the fermentation must is stabilized. A solid/liquid separation step is then performed to separate the cake comprising the biomass from a liquid aqueous phase comprising the vanillin. The solid/liquid separation may be performed via any of the methods described in embodiments 1 to 6. The aqueous liquid phase is then subjected to a step of adsorption on resin or pertraction with simultaneous addition of an organic solvent. The organic phase comprising the vanillin is then subjected to the concentrating, separation of the heavy products and crystallization steps as described in the first embodiment.

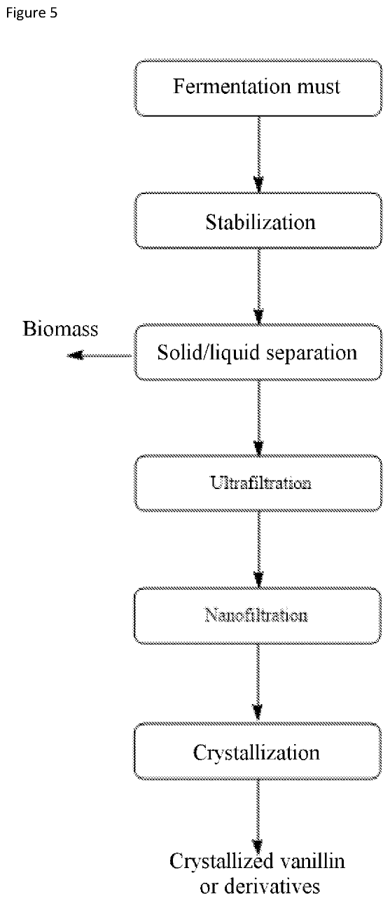

According to a ninth embodiment, represented by FIG. 5, the fermentation must is stabilized. A solid/liquid separation step is then performed so as to separate the biomass; preferably, this separation is performed by microfiltration or diafiltration. This first separation may be followed by an ultrafiltration step. In particular, this ultrafiltration allows the separation of particles not separated out during the ceramic membrane separation. The ultrafiltrate is then subjected to a nanofiltration (or possibly reverse osmosis) step so as to concentrate the vanillin stream. This vanillin stream is then subjected to a crystallization step in a water/alcohol solvent as described previously.

### EXAMPLES

A composition as defined in Table 1 is subjected to crystallization in a water/ethanol mixture, in which the ethanol content is between 15% and 25% by mass. Crystallization is performed by cooling from 40° C. to 4° C. The vanillin concentration at the start of crystallization is between 15% and 25% by mass.

TABLE 1

| | |
|---|---|
| Vanillin (obtained via a | 95% |
| biotechnological process) | |
| Vanillyl alcohol | 2.7% |
| Other compounds | 2.3% |

After crystallization, the crystallized vanillin is filtered off, washed with water, dried and analyzed. The crystallized vanillin has a purity of 99.6%. The vanillyl alcohol content is 0.04%. The vanillic acid content is 0.01%.

Advantageously, the overall yield of vanillin during crystallization is 94% without recycling the crystallization filtrate.

### Example 2 (Comparative)

A composition as defined in Table 1 is subjected to vacuum evaporation.

### Example 2.1

After evaporation, in batch mode, the purified vanillin is analyzed.

The purified vanillin has a purity of 99.7%. The vanillyl alcohol content is less than 0.01%. The vanillic acid content is less than 0.01%.

However, the overall yield of vanillin obtained in Example 2.1 is only 70%.

### Example 2.2

After evaporation, in continuous mode, the purified vanillin is analyzed.

The overall yield of vanillin obtained in Example 2.2 is 95%. However, the purified vanillin has a purity of 98%. The vanillyl alcohol content is 1%. The vanillic acid content is less than 0.01%.

The process of the present invention advantageously allows vanillin to be obtained with good purity characteristics and in very good yields.

1. A process for purifying vanillin or derivatives thereof obtained via a biotechnological process, the process comprising:
at least one step of separating vanillin or derivatives thereof from vanillyl alcohol or derivatives thereof by crystallization.

2. The process of claim 1, characterized in that crystallization is performed in a water/alcohol mixture.

3. The process of claim 1, characterized in that the process further comprises at least one step of stabilizing a fermentation must comprising vanillin or derivatives thereof obtained via a biotechnological process.

4. The process of claim 1, characterized in that the process further comprises at least one biomass separation step.

5. The process of claim 1, characterized in that the process further comprises at least one step of concentrating an aqueous or organic solution of vanillin or derivatives thereof.

6. The process of claim 1, characterized in that the process further comprises at least one step of separating vanillin or derivatives thereof from compounds having a higher boiling point than that of vanillin or derivatives thereof.

7. The process of claim 1, in which the vanillin or derivatives thereof obtained on conclusion of the crystallization step have a color in ethanolic solution at 10% by weight of less than or equal to 150 Hazen.

8. The process of claim 2, in which the amount of alcohol is between 2% and 40% by weight.

9. The process of claim 1, in which the vanillin or derivatives thereof obtained on conclusion of the crystallization step have a color in ethanolic solution at 10% by weight of less than or equal to 100 Hazen.

10. The process of claim 1, in which the vanillin or derivatives thereof obtained on conclusion of the crystallization step have a color in ethanolic solution at 10% by weight of less than or equal to 50 Hazen.
